(19) United States
(12) Patent Application Publication
(10) Pub. No.: US 2024/0285714 A1
(43) Pub. Date: Aug. 29, 2024

(54) COMPOSITION FOR ANTI-INFLAMMATORY, ANTIOXIDANT, ANTI-WRINKLE OR HAIR HEALTH PROMOTION CONTAINING EXTRACT MIXTURE OF ZIZIPHUS JUJUBA MILLER, CITRUS UNSHIU PEEL AND ACORUS CALAMUS AS ACTIVE INGREDIENT

(71) Applicant: INDUSTRY-ACADEMIC COOPERATION FOUNDATION DAEGU HAANY UNIVERSITY, Gyeongsan-si (KR)

(72) Inventors: Jin Han PARK, Daegu (KR); Ki Han KWON, Gyeongsan-si (KR)

(73) Assignee: INDUSTRY-ACADEMIC COOPERATION FOUNDATION DAEGU HAANY UNIVERSITY, Gyeongsan-si (KR)

(21) Appl. No.: 18/400,320

(22) Filed: Dec. 29, 2023

(30) Foreign Application Priority Data
Feb. 28, 2023 (KR) 10-2023-0026578

(51) Int. Cl. A61K 36/725; A61K 8/9789; A61K 8/9794; A61K 36/752; A61K 36/882; A61P 29/00; A61Q 5/00; A61Q 19/08

(52) U.S. Cl. CPC A61K 36/725; A61K 8/9789; A61K 8/9794; A61K 36/752; A61K 36/882; A61P 29/00; A61Q 5/002; A61Q 19/08

(57) ABSTRACT

Provided is a composition for anti-inflammatory, antioxidant, anti-wrinkle or hair health promotion containing an extract mixture of Ziziphus jujuba Miller, Citrus unshiu Peel, and Acorus calamus as an active ingredient. The extract mixture of Ziziphus jujuba Miller, Citrus unshiu Peel, and Acorus calamus decreases the expression of MMP-1 and MMP-13 in fibroblasts and decreases the expression and production of IL-1P and IL-6 by LPS in macrophages to have anti-inflammatory and anti-wrinkle effects. In addition, the extract mixture of Ziziphus jujuba Miller, Citrus unshiu Peel, and Acorus calamus has DPPH radical scavenging activity to have an antioxidant effect. The hair using the extract mixture of Ziziphus jujuba Miller, Citrus unshiu Peel, and Acorus calamus is increased in tensile strength and has an effect of improving a cuticle layer of the hair, thereby having a hair health promotion effect.

### CROSS-REFERENCE TO RELATED APPLICATIONS

This application is based on and claims priority from Korean Patent Application No. 10-2023-0026578, filed on Feb. 28, 2023, with the Korean Intellectual Property Office, the disclosure of which is incorporated herein in its entirety by reference.

### TECHNICAL FIELD

The present disclosure relates to a composition for anti-inflammatory, antioxidant, anti-wrinkle or hair health promotion containing an extract mixture of Ziziphus jujuba Miller, Citrus unshiu Peel and Acorus calamus as an active ingredient.

### BACKGROUND

Skin is a membrane that covers the outside of the body and serves to protect and regulate internal organs and other organs of the body by performing various physiological functions to protect the body from various external environmental factors. The ideal temperature of the skin is 30 to 31° C., which is lower than body temperature, and when the skin is exposed to intense sunlight for 30 minutes, the skin temperature rises up to 43° C., and when the skin is exposed to the environment for a long time, the skin rapidly ages. When the skin temperature rises, the synthesis of collagen as a major component of the skin decreases, and various proteolytic enzymes present in the skin increase. As a result, collagen and elastic fibers are damaged, and the skin balance is broken, so that elasticity is lost and wrinkles occur. As such, the aging phenomenon that occurs when the skin temperature rises is called skin heat aging, and it is important to soothe the skin to prevent such a phenomenon.

Hair grows from hair follicles in the skin and has the characteristic of continuously repeating cycles including a growth phase, a catagen phase, and a resting phase throughout life. It is known that various hormones, cytokines, and growth factors are involved in hair morphogenesis and hair growth regulation, and an increase in the number and size of hair follicles was observed during the growth phase. At this time, the hair follicles in the resting phase are located in the dermis layer, whereas the hair follicles in the growth phase are located deep in the subcutaneous tissue, so that the number and size of hair follicles are closely related to the growth phase.

Ziziphus jujuba Miller has been used as an herbal drug in oriental medicine for a long time due to a high vitamin C content and an anti-aging effect, and is known to have a variety of disease-treating effects, including a cosmetic effect, effects of improving blood circulation, stabilizing the mind, detoxifying various medicinal poisons, an anti-allergy effect, etc.

Currently, a need to develop bioactive substances from natural products such as Ziziphus jujuba Miller is increasing, and methods capable of using these active ingredients safely and hygienically have been researched.

Therefore, the present inventors repeated studies to develop a composition for anti-inflammatory, antioxidant, anti-wrinkle, or hair health promotion using natural products, and as a result, confirmed that Ziziphus jujuba Miller had an anti-inflammatory, antioxidant, anti-wrinkle or hair health promotion effect, and then completed the present disclosure.

### SUMMARY

The present disclosure has been made in an effort to provide a health functional food composition for anti-inflammatory, antioxidant, anti-wrinkle or hair health promotion containing an extract mixture of Ziziphus jujuba Miller, Citrus unshiu Peel, and Acorus calamus as an active ingredient.

The present disclosure has also been made in an effort to provide a pharmaceutical composition for anti-inflammatory, antioxidant, anti-wrinkle or hair health promotion containing an extract mixture of Ziziphus jujuba Miller, Citrus unshiu Peel, and Acorus calamus as an active ingredient.

The present disclosure has also been made in an effort to provide a cosmetic composition for anti-inflammatory, antioxidant, anti-wrinkle or hair health promotion containing an extract mixture of Ziziphus jujuba Miller, Citrus unshiu Peel, and Acorus calamus as an active ingredient.

To achieve the above objectives, the present disclosure provides a health functional food composition for anti-inflammatory, antioxidant, anti-wrinkle or hair health promotion containing an extract mixture of Ziziphus jujuba Miller, Citrus unshiu Peel, and Acorus calamus as an active ingredient.

In an exemplary embodiment of the present disclosure, the composition may be prepared by mixing 55 to 65 wt % of a Ziziphus jujuba Miller extract, 15 to 25 wt % of a Citrus unshiu Peel extract, and 15 to 25 wt % of an Acorus calamus extract, but is not limited thereto.

In an exemplary embodiment of the present disclosure, the Citrus unshiu Peel extract may be prepared by adding distilled water 15 to 25 times as much as Citrus unshiu Peel powder, and then extracting and filtering the mixture at 10 to 30° C. for 20 to 28 hours, but is not limited thereto.

In an exemplary embodiment of the present disclosure, the Acorus calamus extract may be prepared by adding distilled water 15 to 25 times as much as Acorus calamus powder, and then extracting and filtering the mixture at 7 to 100° C. for 20 to 28 hours, but is not limited thereto.

In an exemplary embodiment of the present disclosure, the composition may inhibit the expression of MMP-1 and MMP-13, but is not limited thereto.

In an exemplary embodiment of the present disclosure, the composition may inhibit the expression and production of IL-1β and IL-6, but is not limited thereto.

In an exemplary embodiment of the present disclosure, the composition may increase hair tensile strength, but is not limited thereto.

Another exemplary embodiment of the present disclosure provides a pharmaceutical composition for anti-inflammatory, antioxidant, anti-wrinkle or hair health promotion containing an extract mixture of Ziziphus jujuba Miller, Citrus unshiu Peel, and Acorus calamus as an active ingredient.

Finally, yet another exemplary embodiment of the present disclosure provides a cosmetic composition for anti-inflammatory, antioxidant, anti-wrinkle or hair health promotion containing an extract mixture of Ziziphus jujuba Miller, Citrus unshiu Peel, and Acorus calamus as an active ingredient.

According to the exemplary embodiments of the present disclosure, the extract mixture of Ziziphus jujuba Miller, Citrus unshiu Peel, and Acorus calamus decreases the expression of MMP-1 and MMP-13 in fibroblasts and decreases the expression and production of IL-1β and IL-6 by LPS in macrophages to have anti-inflammatory and anti-wrinkle effects. In addition, the extract mixture of Ziziphus jujuba Miller, Citrus unshiu Peel, and Acorus calamus has DPPH radical scavenging activity to have an antioxidant effect. The hair using the extract mixture of Ziziphus jujuba Miller, Citrus unshiu Peel, and Acorus calamus is increased in tensile strength and has an effect of improving a cuticle layer of the hair, thereby having a hair health promotion effect. By confirming these effects, the extract mixture of Ziziphus jujuba Miller, Citrus unshiu Peel, and Acorus calamus of the present disclosure can be effectively used.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

FIG. 1 is a diagram illustrating cell viability of RAW 264.7 cells according to treatment of an extract mixture of Ziziphus jujuba Miller, Citrus unshiu Peel, and Acorus calamus according to an exemplary embodiment of the present disclosure.

FIG. 2 is a diagram illustrating cell viability of CCD-986sk cells according to treatment of an extract mixture of Ziziphus jujuba Miller, Citrus unshiu Peel, and Acorus calamus according to an exemplary embodiment of the present disclosure.

FIG. 3 is a diagram illustrating mRNA expression levels of IL-1) according to treatment of an extract mixture of Ziziphus jujuba Miller, Citrus unshiu Peel, and Acorus calamus according to an exemplary embodiment of the present disclosure.

FIG. 4 is a diagram illustrating mRNA expression levels of IL-6 according to treatment of an extract mixture of Ziziphus jujuba Miller, Citrus unshiu Peel, and Acorus calamus according to an exemplary embodiment of the present disclosure.

FIG. 5 is a diagram illustrating protein production of IL-1β according to treatment of an extract mixture of Ziziphus jujuba Miller, Citrus unshiu Peel, and Acorus calamus according to an exemplary embodiment of the present disclosure.

FIG. 6 is a diagram illustrating protein production of IL-6 according to treatment of an extract mixture of Ziziphus jujuba Miller, Citrus unshiu Peel, and Acorus calamus according to an exemplary embodiment of the present disclosure.

FIG. 7 is a diagram illustrating mRNA expression levels of MMP-1 and MMP-13 according to treatment of an extract mixture of Ziziphus jujuba Miller, Citrus unshiu Peel, and Acorus calamus according to an exemplary embodiment of the present disclosure.

FIG. 8 is a diagram illustrating DPPH radical scavenging activity of an extract mixture of Ziziphus jujuba Miller, Citrus unshiu Peel, and Acorus calamus according to an exemplary embodiment of the present disclosure.

FIG. 9 is a diagram illustrating cuticle according to treatment of an extract mixture of Ziziphus jujuba Miller, Citrus unshiu Peel, and Acorus calamus according to an exemplary embodiment of the present disclosure.

### DETAILED DESCRIPTION

Hereinafter, an exemplary embodiment of the present disclosure will be described in detail with reference to the accompanying drawings. However, the following exemplary embodiments are presented as examples for the present disclosure, and when it is determined that a detailed description of well-known technologies or configurations known to those skilled in the art may unnecessarily obscure the gist of the present disclosure, the detailed description thereof may be omitted, and the present disclosure is not limited thereto. Various modifications and applications of the present disclosure are possible within the description of claims to be described below and the equivalent scope interpreted therefrom.

Terminologies used herein are terminologies used to properly express exemplary embodiments of the present disclosure, which may vary according to a user, an operator's intention, or customs in the art to which the present disclosure pertains. Accordingly, definitions of the terminologies need to be described based on contents throughout this specification. Throughout the specification, when a part “comprises” a certain component, it is meant that the part may further include other components, not excluding other components, unless explicitly described to the contrary.

Throughout this specification, ‘%’ used to indicate the concentration of a specific material is solid/solid (w/w) %, solid/liquid (w/v) %, and liquid/liquid (v/v) %, unless otherwise stated.

In an aspect, the present disclosure provides a health functional food composition for anti-inflammatory, antioxidant, anti-wrinkle or hair health promotion containing an extract mixture of Ziziphus jujuba Miller, Citrus unshiu Peel, and Acorus calamus as an active ingredient.

The extract according to the present disclosure may be obtained and used by extraction and separation from the nature using extraction and separation methods known in the art, and the “extract” as defined in the present disclosure is extracted from Ziziphus jujuba Miller, Citrus unshiu Peel, and Acorus calamus using a suitable solvent, and includes all extracts, for example, a crude extract, a polar solvent-soluble extract, and a non-polar solvent-soluble extract. The suitable solvent for extracting the extract from Ziziphus jujuba Miller, Citrus unshiu Peel, and Acorus calamus may use any pharmaceutically acceptable organic solvent, and may use water or an organic solvent, but is not limited thereto. For example, various solvents such as purified water, alcohols having carbon atoms 1 to 4 including methanol, ethanol, propanol, isopropanol, butanol, etc., acetone, ether, benzene, chloroform, ethyl acetate, methylene chloride, hexane, cyclohexane, etc. may be used alone or in combination. As an extraction method, any one of methods such as hot water extraction, chilling extraction, reflux cooling extraction, solvent extraction, steam distillation, ultrasonic extraction, elution, and compression may be selected and used. In addition, a desired extract may also be additionally subjected to a conventional fractionation process or may be purified using a conventional purification method.

There is no limitation to a preparation method of the extract of the present disclosure, and any known preparation method may be used. For example, the extract included in the composition of the present disclosure may be prepared with a primary extract extracted by the hot water extraction or solvent extraction method in a powder state by additional processes such as distilling under reduced pressure and freeze-drying or spray-drying. In addition, fractions may also be obtained by further purifying the primary extract using various chromatography, such as silica gel column chromatography, thin layer chromatography, and high performance liquid chromatography. Accordingly, in the present disclosure, the extract is a concept that includes all extracts, and fractioned and purified products that are obtained in each step of extraction, fractionation, or purification, and dilutions, concentrates, or dried products thereof.

In the present disclosure, the composition may be prepared by mixing a Ziziphus jujuba Miller extract of 55 to 65 wt %, preferably 58 to 62 wt %, more preferably 60 wt %, a Citrus unshiu Peel extract of 15 to 25 wt %, preferably 18 to 22 wt %, more preferably 20 wt %, and an Acorus calamus extract of 15 to 25 wt %, preferably 18 to 22 wt %, more preferably 20 wt %, but is not limited thereto.

In the present disclosure, the Citrus unshiu Peel extract may be prepared by adding distilled water 15 to 25 times, preferably 18 to 22 times, more preferably 20 times as much as Citrus unshiu Peel powder, and then extraction and filtration at 10 to 30° C., preferably 15 to 25° C., more preferably 20° C. for 20 to 28 hours, preferably 23 to 25 hours, more preferably 24 hours, but is not limited thereto.

In the present disclosure, the Acorus calamus extract may be prepared by adding distilled water 15 to 25 times, preferably 18 to 22 times, more preferably 20 times as much as Acorus calamus powder, and then extraction and filtration at 10 to 30° C., preferably 15 to 25° C., more preferably 20° C. for 20 to 28 hours, preferably 23 to 25 hours, more preferably 24 hours, but is not limited thereto.

In the present disclosure, the composition may contain an extract mixture of Ziziphus jujuba Miller, Citrus unshiu Peel, and Acorus calamus at a concentration of 0 to 1000 μg/mL, but is limited thereto. However, when the extract mixture is used at a concentration higher than 1000 μg/mL, cytotoxicity occurs or an anti-inflammatory, antioxidant, anti-wrinkle or hair health promotion effect is inhibited, so that it is preferable to use the extract mixture in the concentration range provided by the present disclosure.

In the present disclosure, the composition may inhibit the expression of MMP-1 and MMP-13, but is not limited thereto.

As used herein, the term “MMP” is matrix metalloprotease and accelerates skin aging when exposed to sunlight. The MMP degrades collagen and elastin, which maintain skin elasticity, causing a decrease in collagen content in the skin.

In the present disclosure, the composition may inhibit the expression and production of IL-1β and IL-6, but is not limited thereto.

As used herein, the term “IL-1β” refers to Interleukin-1β, which is one of inflammatory cytokines. IL-1β binds to a specific receptor to activate inflammatory transcription factors, thereby inducing the synthesis and secretion of inflammatory cytokines, including IL-1β, and the secreted inflammatory cytokines serve to induce and sustain an inflammatory response by activating immune cells.

As used herein, the term “IL-6” refers to Interleukin-6, which is one of inflammatory cytokines. IL-6 serves to transmit its biological activity via two types of proteins on the cell membrane.

In the present disclosure, the composition may increase hair tensile strength, but is not limited thereto.

In addition to containing the extract mixture of Ziziphus jujuba Miller, Citrus unshiu Peel, and Acorus calamus as the active ingredient, the food composition of the present disclosure may contain various flavoring agents or natural carbohydrates, or the like as additional ingredients, like conventional food compositions.

Examples of the above-mentioned natural carbohydrates may include general sugars, such as monosaccharides, such as glucose, fructose, etc.; disaccharides, such as maltose, sucrose, etc.; and polysaccharides, such as dextrin, cyclodextrin, etc., and sugar alcohols such as xylitol, sorbitol, erythritol, etc. The above-mentioned flavoring agents may be advantageously used with natural flavoring agents (thaumatin), stevia extracts (e.g., rebaudioside A, glycyrrhizin, etc.), and synthetic flavoring agents (saccharin, aspartame, etc.). The food composition of the present disclosure may be formulated in the same manner as the pharmaceutical composition to be used as functional foods or added to various foods. The foods capable of adding the composition of the present disclosure may include, for example, beverages, meat, chocolate, foods, confectionery, pizza, ramen, other noodles, gums, candies, ice creams, alcohol beverages, vitamin complexes, health food supplements, etc.

In addition, the food composition may contain various nutrients, vitamins, minerals (electrolytes), flavoring agents such as synthetic and natural flavoring agents, coloring agents and enhancers (cheese, chocolate, etc.), pectic acid and salts thereof, alginic acid and salts thereof, organic acid, a protective colloidal thickener, a pH adjusting agent, a stabilizer, a preservative, glycerin, alcohols, a carbonic acid agent used in a carbonated drink, etc., in addition to the extract mixture of Ziziphus jujuba Miller, Citrus unshiu Peel, and Acorus calamus as the active ingredient. In addition, the food composition of the present disclosure may contain pulp for preparing natural fruit juice, fruit juice beverages, and vegetable beverages.

The functional food composition of the present disclosure may be prepared and processed in the form of tablets, capsules, powders, granules, liquids, pills, etc. In the present disclosure, the “health functional food composition” refers to foods prepared and processed by using raw materials or ingredients with functionality, which are useful for the human body according to the Art on Health Functional Foods No. 6727, and means foods taken for adjusting nutrients for the structures and functions of the human body or obtaining a useful effect on health applications such as physiological actions. The health functional food of the present disclosure may include conventional food additives, and the suitability as the food additives is determined by the specifications and standards for the corresponding item in accordance with the general rules of the Food Additive Codex, general test methods, etc. approved by the Food and Drug Administration, unless otherwise specified. The items disclosed in the “Food Additives Codex” may include, for example, chemical composites such as ketones, glycine, calcium citrate, nicotinic acid, cinnamic acid, etc.; natural additives such as desensitizing dye, licorice extract, crystal cellulose, Kaoliang color, guar gum, etc.; mixed formulations such as sodium L-glutamic acid formulations, noodle additive alkali agents, preservative formulations, tar color formulations, etc. For example, the health functional food in the form of tablets may formed by granulating a mixture obtained by mixing the active ingredients of the present disclosure with an excipient, a binder, a disintegrant, and other additives in a conventional manner, and then compression-molding the mixture by adding a slip modifier and the like, or directly compression-molding the mixture. In addition, the health functional food in the form of tablets may also contain a flavor enhancer or the like as needed. In the health functional food in the form of capsules, hard capsules may be prepared by filling a mixture mixed with the active ingredient of the present disclosure and additives such as excipients into conventional hard capsules, and soft capsules may be prepared by filling a mixture mixed with the active ingredient of the present disclosure and additives such as excipients into capsule bases such as gelatin. The soft capsules may contain a plasticizer such as glycerin or sorbitol, a colorant, a preservative, etc., if necessary. The health functional food in the form of pills may be prepared by molding a mixture mixed with the active ingredients of the present disclosure and an excipient, a binder, a disintegrant, etc. by existing known methods, and may also be coated with white sugar or other coating agents or surface-coated with materials such as starch and talc, if necessary. The health functional food in the form of granules may be prepared by granulating a mixture mixed with the active ingredients of the present disclosure and an excipient, a binder, a disintegrant, etc. by existing known methods and may contain a flavoring agent, a flavor enhancer, etc., if necessary.

In another aspect, the present disclosure provides a pharmaceutical composition for anti-inflammatory, antioxidant, anti-wrinkle or hair health promotion containing an extract mixture of Ziziphus jujuba Miller, Citrus unshiu Peel, and Acorus calamus as an active ingredient.

The pharmaceutical composition of the present disclosure may further include an adjuvant in addition to the extract mixture of Ziziphus jujuba Miller, Citrus unshiu Peel, and Acorus calamus as the active ingredient. The adjuvant may be used with any adjuvant known in the art without limitation, but further include, for example, a Freund's complete adjuvant or incomplete adjuvant to increase the immunity thereof.

The pharmaceutical composition according to the present disclosure may be prepared in the form of incorporating the active ingredient of the extract mixture of Ziziphus jujuba Miller, Citrus unshiu Peel, and Acorus calamus into a pharmaceutically acceptable carrier. Here, the pharmaceutically acceptable carrier includes carriers, excipients and diluents commonly used in a pharmaceutical field. The pharmaceutically acceptable carrier that may be used in the pharmaceutical composition of the present disclosure is not limited thereto, but may include lactose, dextrose, sucrose, sorbitol, mannitol, xylitol, erythritol, maltitol, starch, acacia gum, alginate, gelatin, calcium phosphate, calcium silicate, cellulose, methyl cellulose, polyvinylpyrrolidone, water, methyl hydroxybenzoate, propyl hydroxybenzoate, talc, magnesium stearate, and mineral oil.

The pharmaceutical composition of the present disclosure may be formulated and used in the form of oral formulations, such as powders, granules, tablets, capsules, suspensions, emulsions, syrups, aerosols, etc., external preparations, suppositories, or sterile injectable solutions according to each conventional method.

The formulations may be prepared by using diluents or excipients, such as a filler, an extender, a binder, a wetting agent, a disintegrating agent, a surfactant, etc., which are generally used. Solid formulations for oral administration include tablets, pills, powders, granules, capsules, etc., and these solid formulations may be prepared by mixing at least one excipient, for example, starch, calcium carbonate, sucrose, lactose, gelatin, etc. with the active ingredient. Further, lubricants such as magnesium stearate and talc may be used in addition to simple excipients. Liquid formulations for oral administration may correspond to suspensions, oral liquids, emulsions, syrups, etc., and may include various excipients, for example, a wetting agent, a sweetener, an aromatic agent, a preserving agent, etc., in addition to the commonly used simple diluent such as water and liquid paraffin. Formulations for parenteral administration include sterile aqueous solutions, non-aqueous solvents, suspensions, emulsions, lyophilized agents, and suppositories. As the non-aqueous solution and the suspension, propylene glycol, polyethylene glycol, vegetable oil such as olive oil, injectable ester such as ethyl oleate, etc. may be used. As the base of the suppository, witepsol, Tween 61, cacao butter, laurinum, glycerogelatin, etc. may be used.

The pharmaceutical composition of the present disclosure may be administered to a subject through various routes. All methods of administration may be expected, and the pharmaceutical composition may be administered, for example, oral, intravenous, intramuscular, subcutaneous, and intraperitoneal injection.

The dose of the pharmaceutical composition according to the present disclosure is selected in consideration of the age, body weight, sex, and physical condition of the subject. It is obvious that the concentration of the active ingredient included in the pharmaceutical composition may be variously selected according to a subject, and preferably the active ingredient is included in the pharmaceutical composition at a concentration of 0.01 to 5,000 μg/ml. When the concentration is less than 0.01 μg/ml, pharmaceutical activity may not be shown, and when the concentration exceeds 5,000 μg/ml, toxicity to the human body may be exhibited.

The pharmaceutical composition may be formulated into various oral or parenteral dosage forms.

Formulations for oral administration include, for example, tablets, pills, hard and soft capsules, solutions, suspensions, emulsifiers, syrups, granules, etc., and these formulations may further include diluents (e.g., lactose, dextrose, sucrose, mannitol, sorbitol, cellulose and/or glycine), lubricants (e.g., silica, talc, stearic acid and its magnesium or calcium salts and/or polyethylene glycol), in addition to the active ingredient. In addition, the tablets may contain a binder such as magnesium aluminum silicate, starch paste, gelatin, tragacanth, methylcellulose, sodium carboxymethylcellulose and/or polyvinylpyrrolidine, and in some cases, may contain disintegrants or effervescent mixtures such as starch, agar, alginic acid or its sodium salt and/or an absorbent, a coloring agent, a flavoring agent and a sweetening agent. The formulations may be prepared by conventional mixing, granulating or coating methods.

In addition, a typical formulation for parenteral administration is an injection formulation, and as a solvent for the injection formulation, water, a Ringer's solution, isotonic physiological saline or a suspension may be used. Sterile fixed oils of the injection formulation may be used as a solvent or suspension medium, and any non-irritating fixed oil including mono- and di-glycerides may also be used for this purpose.

In addition, as the injection formulation, fatty acids such as oleic acid may be used.

In an aspect, the present disclosure provides a cosmetic composition for anti-inflammatory, antioxidant, anti-wrinkle or hair health promotion containing an extract mixture of Ziziphus jujuba Miller, Citrus unshiu Peel, and Acorus calamus as an active ingredient.

The composition containing the extract mixture of Ziziphus jujuba Miller, Citrus unshiu Peel, and Acorus calamus of the present disclosure may be variously used for hair health promotion, hair growth promotion, etc. Products capable of adding the composition may include, for example, cosmetics such as various creams, lotions, toners, and essences, shampoos, rinses, cleansers, face washes, soaps, treatments, packs, beauty essences, etc.

The cosmetics of the present disclosure may include a composition selected from the group consisting of water-soluble vitamins, oil-soluble vitamins, high-molecular peptides, high-molecular polysaccharides, sphingolipids, and seaweed extracts.

The water-soluble vitamin may include any vitamin that can be blended with cosmetics, but preferably include vitamin B1, vitamin B2, vitamin B6, pyridoxine, pyridoxine hydrochloride, vitamin B12, pantothenic acid, nicotinic acid, nicotinic acid amide, folic acid, vitamin C, vitamin H, etc., and salts (thiamine hydrochloride, sodium ascorbate, etc.) or derivatives (sodium ascorbic acid-2-phosphate, magnesium ascorbic acid-2-phosphate, etc.) thereof are also included in the water-soluble vitamins which may be used in the present disclosure. The water-soluble vitamin may be obtained by conventional methods, such as microbial transformation, purification from a culture solution of a microorganism, enzymatic or chemical synthesis.

The oil-soluble vitamin may include any vitamin that can be blended with cosmetics, but preferably include vitamin A, carotene, vitamin D2, vitamin D3, vitamin E (dl-α tocopherol, d-α tocopherol, d-α tocopherol), etc., and derivatives thereof (ascorbyl palmitate, ascorbyl stearate, ascorbyl dipalmitate, DL-α tocopherol acetate, DL-α tocopherol nicotinic acid vitamin E, DL-pantothenyl alcohol, D-pantothenyl alcohol, pantothenyl ethyl ether, etc.), etc. are also included in the oil-soluble vitamins used in the present disclosure. The oil-soluble vitamins may be obtained by conventional methods, such as microbial transformation, purification from a culture solution of a microorganism, enzymatic or chemical synthesis.

The high-molecular peptide may include any peptide that can be blended with cosmetics, but preferably include collagen, hydrolyzed collagen, gelatin, elastin, hydrolyzed elastin, keratin, etc. The high-molecular peptide may be purified and obtained by conventional methods such as purification from a culture solution of a microorganism, and enzymatic or chemical synthesis, or generally purified and used from natural products such as the dermis of pigs or cattle, fibroin from silkworms, or the like.

The high-molecular polysaccharide may be any polymeric polysaccharide that can be blended with cosmetics, but preferably include hydroxyethyl cellulose, xanthan gum, sodium hyaluronate, chondroitin sulphate or salts thereof (sodium salt, etc.). For example, chondroitin sulphate or salts thereof, etc. may generally be purified and used from mammals or fish.

The sphingolipid may be any sphingolipid that can be blended with cosmetics, but preferably include ceramide, phytosphingosine, glycosphingolipid, etc. The sphingolipid may generally be purified from mammals, fish, shellfish, yeast, or plants by conventional methods or obtained by chemical synthesis.

The seaweed extract may be any seaweed extract that can be blended with cosmetics, but preferably include a brown algae extract, a red algae extract, a green algae extract, etc. In addition, carrageenan, arginic acid, sodium alginate, potassium alginate, etc. purified from these seaweed extracts are also included in the seaweed extract used in the present disclosure. The seaweed extract may be purified and obtained from seaweed by a conventional method.

In addition to the essential ingredients, the cosmetics of the present disclosure may contain other ingredients that may be generally blended with the cosmetics as needed. Other mixed ingredients that may be added herein may include fat ingredients, moisturizers, emollients, surfactants, organic and inorganic pigments, organic powders, UV absorbers, antiseptics, bactericides, antioxidants, plant extracts, pH regulating agents, alcohols, coloring matters, aromatics, blood flow stimulants, cooling agents, antiperspirants, purified water, etc. The fat ingredient may include ester-based fat, hydrocarbon-based fat, silicone-based fat, fluorine-based fat, animal fat, vegetable fat, etc.

The ester-based fat may include glyceryl tri2-ethylhexanoate, cetyl 2-ethylhexanoate, isopropyl myristate, butyl myristate, isopropyl palmitate, ethyl stearate, octyl palmitate, isocetyl isostearate, butyl stearate, ethyl linoleate, isopropyl linoleate, ethyl oleate, isocetyl myristate, isostearyl myristate, isostearyl palmitate, octyldodecyl myristate, isocetyl isostearate, diethyl sebacate, diisopropyl adipate, isoalkyl neopentanoate, glyceryl tricaprylate/tricaprate, trimethylolpropane tri2-ethylhexanoate, trimethylolpropane triisostearate, pentaelislitol tetra2-ethylhexanoate, cetyl caprylate, decyl laurate, hexyl laurate, decyl myristate, myristyl myristate, cetyl myristate, stearyl stearate, decyl oleate, cetyl ricinooleate, isostearyl laurate, isotridecyl myristate, isocetyl palmitate, octyl stearate, isocetyl stearate, isodecyl oleate, octyldodecyl oleate, octyldodecyl linoleate, isopropyl isostearate, cetostearyl 2-ethylhexanoate, stearyl 2-ethylhexanoate, hexyl isostearate, ethylene glycol dioctanoate, ethylene glycol dioleate, propylene glycol dicaprate, propylene glycol dicaprylate/dicaprate, propylene glycol dicaprylate, neopentyl glycol dicaprate, neopentyl glycol dioctanate, glyceryl tricaprylate, glyceryl triundecylate, glyceryl triisopalmitate, glyceryl triisostearate, octyldodecyl neopentanoate, isostearyl octanoate, octyl isononanoate, hexyldecyl neodecanoate, octyldodecyl neodecanoate, isocetyl isostearate, isostearyl isostearate, octyldecyl isostearate, polyglycerol oleate ester, polyglycerol isostearate ester, triisocetyl citrate, triisoalkyl citrate, triisooctyl citrate, lauryl lactate, myristyl lactate, cetyl lactate, octyldecyl lactate, triethyl citrate, acetyl triethyl citrate, acetyl tributyl citrate, trioctyl citrate, diisostearyl malate, 2-ethylhexyl hydroxystearate, di2-ethylhexyl succinate, diisobutyl adipate, diisopropyl sebacate, dioctyl sebacate, cholesteryl stearate, cholesteryl isostearate, cholesteryl hydroxystearate, cholesteryl oleate, dihydrocholesteryl oleate, phytosteryl isostearate, phytosteryl oleate, isocetyl 12-stealoyl hydroxystearate, stearyl 12-stealoyl hydroxystearate, isostearyl 12-stealoyl hydroxystearate, etc.

The hydrocarbon-based fat may include squalene, liquid paraffin, α-olefin oligomer, isoparaffin, ceresin, paraffin, liquid isoparaffin, polybudene, microcrystalline wax, Vaseline, etc.

The silicone-based fat may include polymethyl silicone, methylphenyl silicone, methylcyclopolysiloxane, octamethylpolysiloxane, decamethylpolysiloxane, dodecamethylcyclosiloxane, a dimethylsiloxane/methylcetyloxysiloxane copolymer, a dimethylsiloxane/methylstealoxysiloxane copolymer, alkyl-modified silicone oil, amino-modified silicone oil, etc.

The fluorine-based fat may include perfluoropolyether, etc.

The animal or vegetable fat may include animal or vegetable fat, such as avocado oil, almond oil, olive oil, sesame oil, rice bran oil, safflower seed oil, soybean oil, corn oil, rapeseed oil, apricot seed oil, palm kernel oil, palm oil, castor oil, sunflower oil, grape seed oil, cottonseed oil, coconut oil, candlenut oil, wheat germ oil, rice germ oil, shea butter, evening primrose oil, macadamia nut oil, meadowfoam oil, egg yolk oil, beef tallow, horse oil, mink oil, orange roughy oil, jojoba oil, candelilla wax, carnauba wax, liquid lanolin, hydrogenated castor oil, etc.

The moisturizer may include a water-soluble low-molecular moisturizer, a fat-soluble molecular moisturizer, a water-soluble polymer, a fat-soluble polymer, and the like.

The water-soluble low-molecular moisturizer may include serine, glutamine, sorbitol, mannitol, sodium pyrrolidone-carboxylate, glycerin, propylene glycol, 1,3-butylene glycol, ethylene glycol, polyethylene glycol B (degree of polymerization n=2 or more), and polypropylene glycol (degree of polymerization n=2 or more), polyglycerin B (degree of polymerization n=2 or more), lactic acid, lactate, etc.

The fat-soluble low-molecular moisturizer may include cholesterol, cholesterol esters, etc.

The water-soluble polymer may include carboxyvinyl polymer, polyaspartate, tragacanth, xanthan gum, methyl cellulose, hydroxymethyl cellulose, hydroxyethyl cellulose, hydroxypropyl cellulose, carboxymethyl cellulose, water-soluble chitin, chitosan, dextrin, etc.

The fat-soluble polymer may include a polyvinylpyrrolidone/eicosene copolymer, a polyvinylpyrrolidone/hexadecene copolymer, nitrocellulose, dextrin fatty acid ester, polymer silicone, etc. The emollient may include long-chain acyl glutamic acid cholesteryl ester, hydroxystearic acid cholesteryl, 12-hydroxystearic acid, stearic acid, rosin acid, lanolin fatty acid cholesteryl ester, etc.

The surfactant may include a non-ionic surfactant, an anionic surfactant, a cationic surfactant, an amphoteric surfactant, etc.

The non-ionic surfactant may include self-emulsified monostearic acid glycerine, propylene glycol fatty acid ester, glycerine fatty acid ester, polyglycerine fatty acid ester, sorbitan fatty acid ester, polyoxyethylene (POE) sorbitan fatty acid ester, POE sorbite fatty acid ester, POE glycerine fatty acid ester, POE alkylether, POE fatty acid ester, POE hydrogenated caster oil, POE castor oil, a polyoxyethylene (POE)/polyoxypropylene (POP) copolymer, POE/POP alkylether, polyether-modified silicone, lauric acid alkanol amide, alkylamine oxide, hydrogenated soybean phospholipid, etc.

The anionic surfactant may include fatty acid soap, α-acylsulfonate, alkylsulfonate, alkylarylsulfonate, alkylnaphthalenesulfonate, alkylsulfate, POE alkylethersulfate, alkylamidesulfate, alkylphosphate, POE alkylphosphate, alkylamidephosphate, alkyloylalkyltaurate, N-acylamino acid salt, POE alkylethercarboxylate, alkylsulfosuccinate, sodium alkylsulfoacetate, acylated hydrolyzed collagen peptide salt, perfluoroalkyl phosphate ester, etc.

The cationic surfactant may include alkyltrimethyl ammonium chloride, stearyltrimethyl ammonium chloride, stearyltrimethylammonium bromide, cetostearyltrimethyl ammonium chloride, distearyldimethyl ammonium chloride, stearyldimethylbenzyl ammonium chloride, behenyltrimethyl ammonium bromide, benzalkonium chloride, diethylaminoethylamide stearate, dimethylaminopropylamide stearate, quaternary ammonium derivatives of lanolin, etc. The amphoteric surfactant may include carboxybetaine-type, amide betaine-type, sulfobetaine-type, hydroxyl sulfobetaine-type, amide sulfobetaine-type, phosphobetaine-type, aminocarboxylate-type, imidazoline derivative-type, amideamine-type amphoteric surfactants, etc.

The organic and inorganic pigments may include an inorganic pigment such as silicic acid, silica, magnesium silicate, talc, sericite, mica, kaolin, rouge, clay, bentonite, titan-coated mica, bismuth oxychloride, zirconium oxide, magnesium oxide, zinc oxide, titanium oxide, aluminium oxide, calcium sulfate, barium sulfate, magnesium sulfate, calcium carbonate, magnesium carbonate, iron oxide, ultramarine, chromium oxide, chromium hydroxide, calamine and a complex thereof; an organic pigment such as polyamide, polyester, polypropylene, polystyrene, polyurethane, vinyl resin, urea resin, phenol resin, fluorine resin, silica resin, acrylic resin, melamine resin, epoxy resin, polycarbonate resin, a divinylbenzene/styrene copolymer, silk powder, cellulose, CI Pigment Yellow, or CI Pigment Orange; and a complex pigment of the inorganic pigment and the organic pigment, etc.

The organic powder may include a metallic soap such as calcium stearate; a metal alkylphosphate such as zinc sodium cetylate, zinc laurylate or calcium laurylate; a polyvalent acylamino acid metal salt such as N-lauroyl-β-alanine calcium, N-lauroyl-β-alanine zinc or N-lauroyl glycine calcium; a polyvalent amide sulfonic acid metal salt such as N-lauroyl-taurine calcium or N-palmitoyl-taurine calcium; a N-acyl basic amino acid such as N-ε-lauroyl-L-lysine, N-ε-palmitoyllysine, N-α-palmitoyl ornithine, N-α-lauroylarginine, or N-α-hydrogenated tallow fatty acid acyl arginine; an N-acylpolypeptide such as N-lauroylglycylglycine; an α-amino fatty acid such as α-amino caprylic acid, or α-amino lauric acid; polyethylene, polypropylene, nylon, polymethylmethacrylate, polystyrene, divinylbenzene/styrene copolymer, tetrafluoroethylene, etc.

The UV absorber may include para-amino benzoic acid, ethyl-para-aminobenzoate, amyl-para-aminobenzoate, octyl-para-aminobenzoate, salicylic acid ethylene glycol, phenyl salicylate, octyl salicylate, benzyl salicylate, butylphenyl salicylate, homomentyl salicylate, benzyl cinnamate, 2-ethoxyethyl para-methoxy cinnamate, octyl para-methoxy cinnamate, mono-2-ethyl hexane glyceryl di-para-methoxy cinnamate, isopropyl para-methoxy cinnamate, a diisopropyl/diisopropyl cinnamic acid ester mixture, urocanic acid, ethyl urocanate, hydroxy methoxybenzophenone, hydroxyl methoxybenzophenone sulfonic acid and slats thereof, dihydroxy methoxybenzophenone, sodium dihydroxy methoxybenzophenone disulfonate, dihydroxy benzophenone, tetrahydroxy benzophenone, 4-tert-butyl-4′-methoxy dibenzoylmethane, 2,4,6-trianilino-p-(carbo-2′-ethylhexyl-1′-oxy)-1,3,5-triazine, 2-(2-hydroxy-5-methylphenyl)benzotriazole, etc.

The bactericide may include hinokitiol, triclosan, trichlorohydroxydiphenyl ether, chlorhexidine gluconate, phenoxy ethanol, resorcin, isopropylmethylphenol, azulene, salicylic acid, zinc pyrithione, benzalkonium chloride, photosensitizer 301, sodium mononitroguaiacol, undecylenic acid, etc.

The antioxidant may include butylhydroxy anisole, propyl gallate, erythorbic acid, etc.

The pH regulating agent may include citric acid, sodium citrate, malic acid, sodium malate, fumaric acid, sodium fumarate, succinic acid, sodium succinate, sodium hydroxide, sodium monohydrogen phosphate, etc.

The alcohol may include a higher alcohol such as cetyl alcohol.

In addition, a blending ingredient which may be added herein is not limited thereto, and any ingredient may also be blended in a range that the objects and effects of the present disclosure are not hindered.

The cosmetics of the present disclosure may be prepared in the form of solutions, emulsions or viscous mixtures.

The ingredients included in the cosmetic composition of the present disclosure may include ingredients which are generally used in the cosmetic composition as active ingredients, and for example, include a conventional adjuvant and carrier such as a stabilizing agent, a solubilizing agent, vitamins, a pigment and an aromatic.

The cosmetic composition for skin whitening and skin anti-aging of the present disclosure may be prepared in any formulation which is generally prepared in the art, and examples thereof may include emulsions, creams, toner, packs, foundations, lotions, essences, hair care cosmetics, etc.

Specifically, the cosmetic composition of the present disclosure includes formulations of skin lotion, skin softener, skin toner, milky lotion, astringent, lotion, moisture lotion, nutritional lotion, massage cream, nutritional cream, moisture cream, hand cream, foundation, essence, nutritional essence, pack, soap, cleansing foam, cleansing lotion, cleansing cream, hair lotion, hair tonic, hair essence, hair shampoo, hair rinse, hair treatment, body lotion and body cleanser.

When the formulation of the present disclosure is the paste, cream, or gel, as a carrier ingredient, animal fiber, vegetable fiber, wax, paraffin, starch, tragacanth, cellulose derivatives, polyethylene glycol, silicone, bentonite, silica, talc, zinc oxide or the like may be used.

When the formulation of the present disclosure is the powder or spray, as the carrier ingredient, lactose, talc, silica, aluminum hydroxide, calcium silicate or polyamide powder may be used, and particularly, in the case of the spray, a propellant such as chlorofluorohydrocarbon, propane/butane or dimethyl ether may be additionally included.

When the formulation of the present disclosure is the solution or emulsion, as the carrier ingredient, a solvent, a solubilizing agent or an emulsifying agent may be used. For example, the carrier ingredient includes water, ethanol, isopropanol, ethyl carbonate, ethyl acetate, benzyl alcohol, benzyl benzoate, propylene glycol, 1,3-butyl glycol oil, glycerol aliphatic ester, polyethylene glycol or sorbitan fatty acid ester.

When the formulation of the present disclosure is the suspension, as the carrier ingredient, a liquid diluent such as water, ethanol or propylene glycol, a suspension such as ethoxylated isostearyl alcohol, polyoxyethylene sorbitol ester and polyoxyethylene sorbitan ester, microcrystalline cellulose, aluminum metahydroxide, bentonite, agar, tragacanth, or the like may be used.

When the formulation of the present disclosure is the surfactant-containing cleansing, as the carrier ingredient, aliphatic alcohol sulfate, aliphatic alcohol ether sulfate, sulfosuccinic acid monoester, isethionate, imidazolinium derivatives, methyltaurate, sarcosinate, fatty acid amide ether sulfate, alkylamido betaine, aliphatic alcohol, fatty acid glyceride, fatty acid diethanolamide, vegetable oil, lanolin derivatives, ethoxylated glycerol fatty acid ester, or the like may be used.

As described above, the specific exemplary embodiments of the present disclosure have been described, but those skilled in the art understanding the spirit of the present disclosure will be able to easily propose other degenerate inventions or other exemplary embodiments included in the scope of the present disclosure by adding, changing, and deleting other elements within the same technical scope. Therefore, it should be appreciated that the exemplary embodiments described above are illustrative in all aspects and are not restricted. The scope of the present disclosure is represented by claims to be described below rather than the detailed description, and it is to be interpreted that the meaning and scope of the claims and all the changes or modified forms derived from the equivalents thereof come within the scope of the present disclosure.

### <Preparation Example 1> Preparation of Ziziphus jujuba Miller Extract

3 kg of Ziziphus jujuba Miller was washed thoroughly in running water, dehydrated, then the seeds and flesh were separated, and ground with a hood mixer for 1 minute. The ground Ziziphus jujuba Miller was added with 13 L of distilled water, boiled for 10 hours, and then filtered three times through a 20 mesh sieve and a cotton cloth to prepare a Ziziphus jujuba Miller extract. The Ziziphus jujuba Miller extract was used and refrigerated at 4° C.

The Ziziphus jujuba Miller extract was prepared as an extract by purchasing a Natural Tree Ziziphus jujuba Miller concentrate from Natural Tree.

### <Preparation Example 2> Preparation of Citrus unshiu Peel Extract

1 g of Citrus unshiu peel powder was added with distilled water at a 20-fold weight ratio, and extracted at 20° C. for 24 hours. The extract was centrifuged three times to obtain a supernatant, and the supernatant was filtered through filter paper to prepare a Citrus unshiu Peel extract. The Citrus unshiu Peel extract was refrigerated at 4° C. and used.

### <Preparation Example 3> Preparation of Acorus calamus Extract

1 g of Acorus calamus powder was added with distilled water at a 20-fold weight ratio, and extracted at 85° C. for 24 hours. The extract was centrifuged three times to obtain a supernatant, and the supernatant was filtered through filter paper to prepare an Acorus calamus extract. The Acorus calamus extract was refrigerated at 4° C. and used.

### <Example 1> Preparation of Extract Mixture of Ziziphus jujuba Miller, Citrus unshiu Peel, and Acorus calamus

An extract mixture of Ziziphus jujuba Miller, Citrus unshiu Peel, and Acorus calamus was prepared by mixing 20 wt % of the Citrus unshiu Peel extract, 20 wt % of the Acorus calamus extract, and 60 wt % of the Ziziphus jujuba Miller extract prepared in Preparation Examples 1 to 3.

The mixture CJD used in the present disclosure was frozen in a −80° C. deep freezer (NIHON FREEZER, Japan), dried in a freeze dryer (OPERON, Gyeonggi-do, Republic of Korea), and then ground and prepared in a powder form. The extract in the powder form was dissolved in distilled water and filtered.

### <Example 2> Cell Culture

A RAW 264.7 murine macrophage cell line was purchased from the Korea Cell Line Bank (Seoul, Republic of Korea). Cells were cultured in DMEM (Dulbecco's-modified Eagle's medium; Gibco, U.S.A) containing 10% FBS (Fetal Bovine Serum; Welgene, Republic of Korea) and 1% Penicillin-streptomycin (10,000 U/mL) (Gibco, U.S.A.) at 37° C. under 5% CO₂ conditions.

CCD-986sk human fibroblasts were purchased from the Korea Cell Line Bank (Seoul, Republic of Korea). Cells were cultured in IMDM (Iscove's Modified Dulbecco's Medium; Lonza Bioscience, U.S.A.) containing 10% FBS and 1% Penicillin-streptomycin (10,000 U/mL) at 37° C. under 5% CO₂ conditions.

### <Experimental Example 1> Cell Viability Analysis

### 1-1. Confirmation of RAW 264.7 Cell Viability

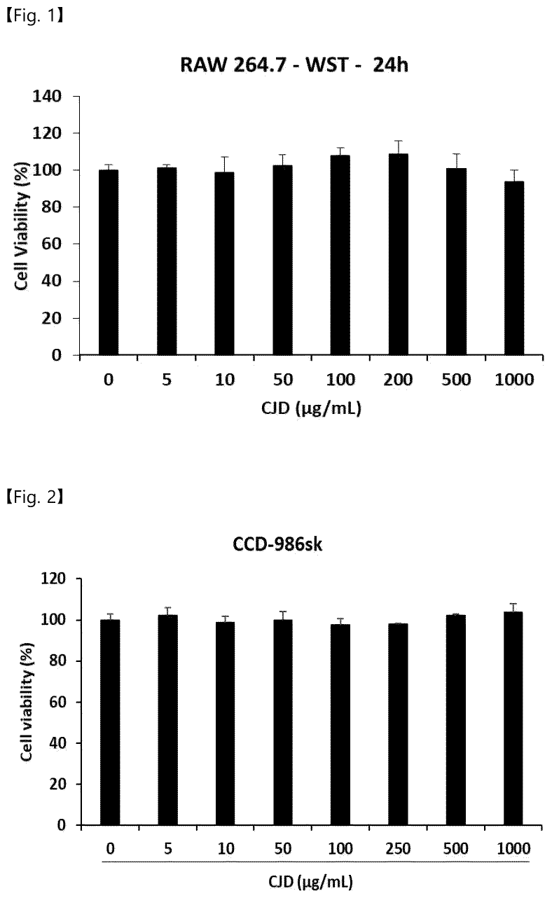

RAW 264.7 cells (5×10³ cells/well), which were macrophages seeded in a 96 well plate, were treated with a drug at different concentrations of 0, 5, 10, 50, 100, 200, 500, and 1000 μg/mL, and after 24 hours, treated with a WST reagent (EZ-Cytox; DoGenBio, Republic of Korea), and then cultured for 2 hours at 37° C. under 5% CO₂ conditions. After culturing, the absorbance was measured at 450 nm using an absorbance meter (Molecular Devices, U.S.A.) and illustrated in FIG. 1.

As illustrated in FIG. 1, it was confirmed that there was no significant decrease in cell viability of macrophages RAW 264.7 depending on the treatment with the extract mixture of Ziziphus jujuba Miller, Citrus unshiu Peel, and Acorus calamus for each concentration. When used at 1000 μg/mL or more, the cytotoxicity was exhibited, so that it is preferable to use the extract mixture of Ziziphus jujuba Miller, Citrus unshiu Peel, and Acorus calamus at a concentration of 1000 μg/mL or less.

### 1-2. Confirmation of CCD-986sk Cell Viability

CCD-986sk cells (1×10⁴ cells/well), which were fibroblasts seeded in a 96 well plate, were treated with a drug at different concentrations of 0, 5, 10, 50, 100, 200, 500, and 1000 μg/mL, and after 48 hours, treated with a WST reagent (EZ-Cytox; DoGenBio, Republic of Korea), and then cultured at 37° C. under 5% CO₂ conditions for 2 hours. After culturing, the absorbance was measured at 450 nm using an absorbance meter (Molecular Devices, U.S.A.) and illustrated in FIG. 2.

As illustrated in FIG. 2, it was confirmed that there was no significant decrease in cell viability of the CCD-986sk cells depending on the treatment with the extract mixture of Ziziphus jujuba Miller, Citrus unshiu Peel, and Acorus calamus for each concentration. When used at 1000 μg/mL or more, the cytotoxicity was exhibited, so that it is preferable to use the extract mixture of Ziziphus jujuba Miller, Citrus unshiu Peel, and Acorus calamus at a concentration of 1000 μg/mL or less.

### <Experimental Example 2> Anti-Inflammatory Efficacy Analysis

2-1. Measurement of mRNA Expression Levels of Proinflammatory Cytokines IL-1β and IL-6

A RAW 264.7 cell line, which was macrophages, was dispensed at 5×10⁵ cells/well in a 6-well plate, and treated with samples at concentrations of 250, 500, and 1000 μg/mL for 1 hour. Thereafter, the cells were treated with 100 ng/mL of lipopolysaccharide (LPS) and cultured at 37° C. under 5% CO₂ conditions for 24 hours. After 24 hours, the supernatant was washed once with PBS, and total RNA was extracted using an RNA spin total RNA extraction kit (iNtRON BioTech, Seoul, Republic of Korea). Quantification was performed using Thermo Scientific™ NanoDrop™ 2000/2000c Spectrophotometers (Thermo Fischer, U.S.A.), and cDNA was synthesized using an iScript cDNA Synthesis kit (Bio-rad, U.S.A.). After synthesis, mRNA expression of IL-1β and IL-6 samples for each concentration was confirmed based on GAPDH mRNA using real-time PCR systems (Applied Biosystems, Foster City, CA, U.S.A.). Primer sequences for IL-1β, IL-6, and GAPDH were shown in Table 1 below.

TABLE 1

| Primer | Base sequence information |
|---|---|
| IL-1β | 5′-CAC AGC AGC ACA TCA ACA AG-3′ |
| | 5′-GTG CTC ATG TCC TCA TCC TG-3′ |
| IL-6 | 5′-CTC TGG GAA ATC GTG GAA AT-3′ |
| | 5′-CCA GTT TGG TAG CAT CCA TC-3′ |
| GAPDH | 5′-GAC ATG CCG CCT GGA GAA AC-3′ |
| | 5′-AGC CCA GGA TGC CCT TTA GT-3′ |

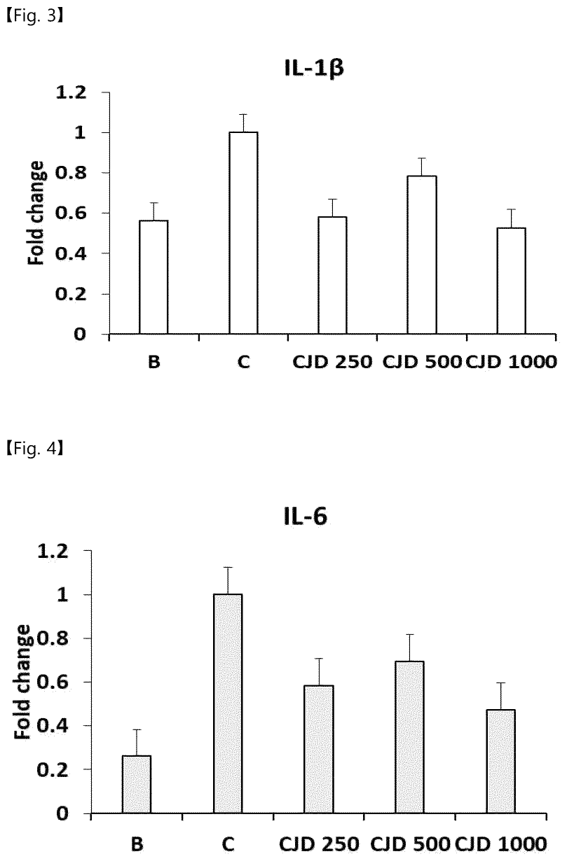

The measured mRNA expression level of IL-1β was illustrated in FIG. 3. In FIG. 3, both the extract and lipopolysaccharide (LPS) were not used as a control group (blank, B), and as a control group (control, C), no extract was used, but only lipopolysaccharide (LPS) was used.

As illustrated in FIG. 3, it was confirmed that the mRNA expression level of IL-1β was decreased at all concentrations compared to the control group. It was confirmed that when the concentration of the extract mixture was 250 μg/mL and 1000 μg/mL, the mRNA expression level was similar to that of the control group (blank), and when the concentration was 1000 μg/mL, IL-1β was expressed the least.

The measured mRNA expression level of IL-6 was illustrated in FIG. 4. As illustrated in FIG. 4, it was confirmed that the mRNA expression level of IL-6 was decreased at all concentrations compared to the control group. It was confirmed that when the concentration of the extract mixture was 1000 μg/mL, IL-6 was expressed the least.

As a result, it was confirmed that the treatment with the extract mixture of Ziziphus jujuba Miller, Citrus unshiu Peel, and Acorus calamus reduced the expression of IL-1β and IL-6 by LPS in macrophages, and thus, had an anti-inflammatory effect.

### 2-2. Measurement of Protein Production of Proinflammatory Cytokines IL-1β and IL-6

A RAW 264.7 cell line, which was macrophages, was dispensed at 5×10⁵ cells/well in a 6-well plate, and treated with samples at concentrations of 250, 500, and 1000 μg/mL for 1 hour. Thereafter, the cells were treated with 100 ng/mL of LPS and cultured at 37° C. under 5% CO₂ conditions for 24 hours. After 24 hours, IL-6 was measured in the supernatant using the BD OptEIA ELISA Kit (BD Pharmingen, San Diego, CA, U.S.A.). A 96-well microplate was coated with a capture antibody overnight at 4° C. and then washed with a washing buffer. The 96-well microplate was blocked with an Assay Diluent for 1 hour, and then washed again 3 times with a washing buffer. A standard and samples were added to each well, incubated at room temperature for 2 hours, and then washed 5 times with a washing buffer. Each well was added with a working detector mixed with a detection antibody and a streptavidin SAv-HRP reagent, reacted for 1 hour, and then washed 7 times with a washing buffer. Each well was added with a substrate solution, reacted in a dark room for 30 minutes, and then added with a stop solution, and the absorbance was measured at 450 nm with an absorbance meter (Molecular Devices, U.S.A.).

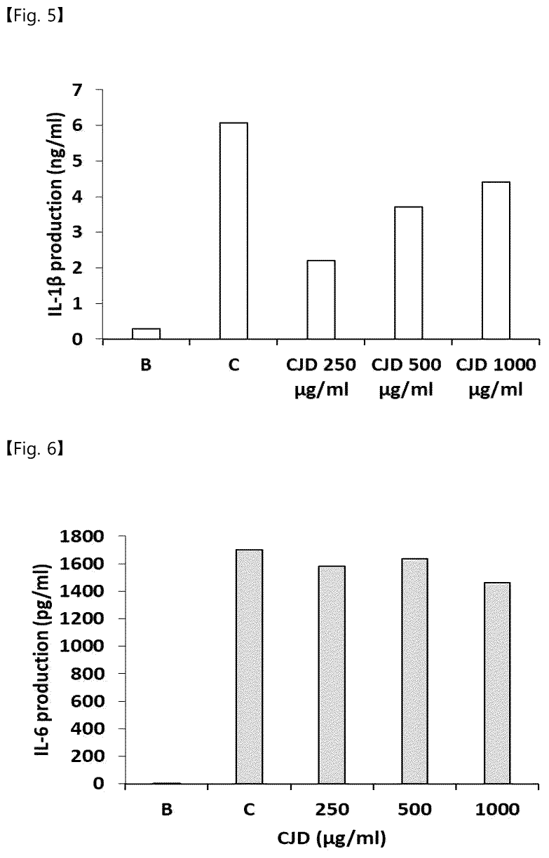

For IL-1, a Mouse IL-1 beta/IL-1F2 Quantikine ELISA kit (R&D system, Abingdon, UK) was used. Each well was added with 50 μl of Assay Diluent RD1-52 and 50 μl of the standard and samples, reacted for 2 hours, and then washed with a washing buffer. Each well was added with 100 μl of a mouse IL-1β conjugate, reacted for 2 hours, and then washed with a washing buffer. Each well was added with 100 μl of a substrate solution, reacted in the dark room, and then added with 100 μl of a stop solution, and the absorbance was measured at 450 nm with an absorbance meter (Molecular Devices, U.S.A) and illustrated in FIGS. 5 and 6.

As illustrated in FIG. 5, it was confirmed that the protein production of IL-1β was low at all concentrations compared to the control group. It was confirmed that when the concentration of the extract mixture was 250 μg/mL, IL-6 was expressed the least.

As illustrated in FIG. 6, the protein production of IL-6 was 1700.13 μg/mL in the control group, 1580.226 μg/mL in a 250 μg/mL group of the extract mixture of Ziziphus jujuba Miller, Citrus unshiu Peel, and Acorus calamus, 1636.949 μg/mL in a 500 μg/mL group of the extract mixture of Ziziphus jujuba Miller, Citrus unshiu Peel, and Acorus calamus, and 1462.01 μg/mL in a 1000 μg/mL group of the extract mixture of Ziziphus jujuba Miller, Citrus unshiu Peel, and Acorus calamus. As a result, it was confirmed that the production of IL-6 was low at all concentrations compared to the control group, and that IL-6 was expressed the least when the concentration of the extract mixture was 1000 μg/mL.

As a result, it was confirmed that the treatment with the extract mixture of Ziziphus jujuba Miller, Citrus unshiu Peel, and Acorus calamus reduced the production of IL-1β and IL-6 by LPS in macrophages, and thus, had an anti-inflammatory effect.

### <Experimental Example 3> Anti-Wrinkle Efficacy Analysis

3-1. Measurement of mRNA Expression Levels of MMP-1 and MMP-13

A CCD 986sk cell line, which was fibroblasts, was dispensed at 1×10⁴ cells/well in a 96-well plate, and treated with samples at concentrations of 250, 500, and 1000 μg/mL for 48 hours. The supernatant was stored separately for ELISA, and total RNA of the cells was extracted using an RNA spin total RNA extraction kit (iNtRON BioTech, Seoul, Repulic of Korea). Quantification was performed using Thermo Scientific™ NanoDrop™ 2000/2000c Spectrophotometers (Thermo Fischer, U.S.A.), and cDNA was synthesized using an iScript cDNA Synthesis kit (Bio-rad, U.S.A.). After synthesis, mRNA expression of Matrix Metalloproteinase-1 (MMP-1) and Matrix Metalloproteinase-13 (MMP-13) samples for each concentration was confirmed based on GAPDH mRNA using real-time PCR systems (Applied Biosystems, Foster City, CA, U.S.A.). Primer sequences of MMP-1 and MMP-13 were shown in Table 2 below.

TABLE 2

| Primer | Base sequence information |
|---|---|
| MMP-1 | TGGACCAGCAATTTCCAAGAT |
| | TCCAAGGGAATGGCCAAA |
| MMP-13 | AGGTAGCGCTCTGCAAACTGG |
| | AGCTGGACTCATTGTCGGGC |

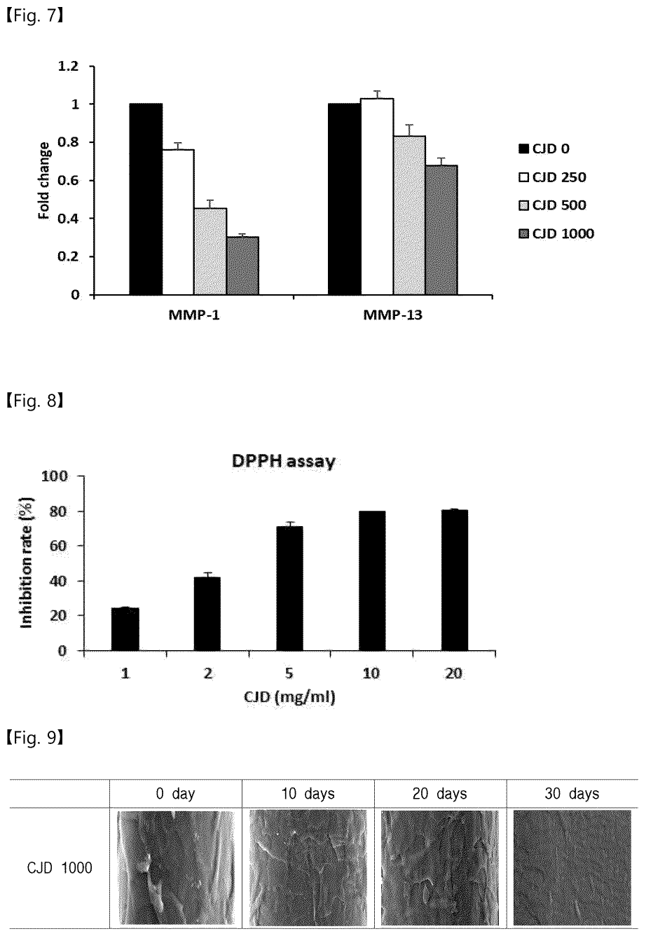

The measured mRNA expression levels of MMP-1 and MMP-13 were illustrated in FIG. 7. As illustrated in FIG. 7, it was confirmed that the mRNA expression levels of MMP-1 and MMP-13 were decreased in a concentration-dependent manner according to treatment with the extract mixture of Ziziphus jujuba Miller, Citrus unshiu Peel, and Acorus calamus.

As a result, it was confirmed that the treatment with the extract mixture of Ziziphus jujuba Miller, Citrus unshiu Peel, and Acorus calamus reduced the expression of MMP-1 and MMP-13 in fibroblasts, and the expression of MMP-1 and MMP-13 was all expressed the least when the concentration of the extract mixture was 1000 μg/mL.

These results mean that the treatment with the extract mixture of Ziziphus jujuba Miller, Citrus unshiu Peel, and Acorus calamus has an anti-wrinkle effect.

### <Experimental Example 4> Antioxidant Efficacy Analysis

### 4-1. Confirmation of DPPH Radical Scavenging Activity

The DPPH radical scavenging activity was confirmed using a 2,2-diphenyl-1-picrylhydrazyl (DPPH) measurement kit (Dojindo, Japan). The DPPH was dissolved in 10 mL of ethanol, and a Trolox standard was prepared for each concentration. Each well was added with 20 μl of the samples at each concentration of 0.5, 1, 2, 5 mg/mL, Blanks 1 and 2 were added with 20 μl of distilled water as a solvent, and then all wells were added with 80 μl of an assay buffer. Blank 2 was added with 100 μl of ethanol, and the remaining wells were added with 100 μl of a DPPH working solution, and reacted at 25° C. in dark conditions for 30 minutes. Absorbance was measured at 517 nm with an absorbance meter (Molecular Devices, USA). Antioxidant efficacy was calculated using Equation 1 below.

$$DPPH radical scavenging activity ( \% ) = \frac{( Acs - As )}{Acs} \times 100 \qquad [ Equation 1 ]$$

Acs: Blank 1—Blank 2

As: Absorbance of samples—Blank 2

As illustrated in FIG. 8, it was confirmed that the extract mixture of Ziziphus jujuba Miller, Citrus unshiu Peel, and Acorus calamus increased the DPPH radical scavenging activity in a concentration-dependent manner.

As a result, the DPPH radical scavenging activity of the extract mixture of Ziziphus jujuba Miller, Citrus unshiu Peel, and Acorus calamus was confirmed, which meant that the extract mixture of Ziziphus jujuba Miller, Citrus unshiu Peel, and Acorus calamus had antioxidant efficacy.

### <Experimental Example 5> Hair Experiment

### 5-1. Measurement of Hair Tensile Strength

The measurement of hair tensile strength was performed to evaluate hair tensile strength with or without treatment of the extract mixture of Ziziphus jujuba Miller, Citrus unshiu Peel, and Acorus calamus.

The hair tensile strength was measured using a universal testing machine (TO-101, Testone, Korea), and a measurement rate was 10 mm/min, and a gauge length was 20 mm. Ten extract mixture samples of Ziziphus jujuba Miller, Citrus unshiu Peel, and Acorus calamus were prepared at concentrations of 250, 500, and 1000 μg/mL and the hair tensile strength was measured 10 times repeatedly. The measured hair tensile strength was shown in Table 3 below.

TABLE 3

| Group | 0 day | 30 days | N |
|---|---|---|---|
| CJD 250 | 73.56 12.78 | 74.25 18.83 | 10 |
| CJD 500 | 79.84 13.21 | 81.39 13.21 | 10 |
| CJD 1000 | 85.67 10.66 | 87.92 12.31 | 10 |

As illustrated in FIG. 3, it was confirmed that the extract mixture of Ziziphus jujuba Miller, Citrus unshiu Peel, and Acorus calamus increased the hair tensile strength in a concentration-dependent manner. It was confirmed that the hair tensile strength treated with the extract mixture of Ziziphus jujuba Miller, Citrus unshiu Peel, and Acorus calamus for 30 days was higher than the hair tensile strength non-treated with the extract mixture of Ziziphus jujuba Miller, Citrus unshiu Peel, and Acorus calamus.

### 5-2. Cuticle Analysis Using SEM

For cuticle analysis, a hair sample was cut into 5 mm long and ion-coated (Au, Pt—Pd sputtering) at a thickness of about 20 mm using a vacuum ion coater (Ion Sputter: E-1020, Hitachi, Japan). The hair was treated with 1000 μg/mL of the extract mixture of Ziziphus jujuba Miller, Citrus unshiu Peel, and Acorus calamus for 30 days, and morphological changes in hair were observed at 10-day intervals using a scanning electron microscope (SEM; JSM-6400, JEOL, Japan). The observed result was illustrated in FIG. 9.

As shown in FIG. 9, it was confirmed that as the period of treatment with the extract mixture of Ziziphus jujuba Miller, Citrus unshiu Peel, and Acorus calamus has passed, the cuticle layers were firmly attached to each other and the surface of the hair became smooth.

As a result, the tensile strength of the hair increased in the hair experiment of the extract mixture of Ziziphus jujuba Miller, Citrus unshiu Peel, and Acorus calamus, and the cuticle layer was improved through SEM analysis of the hair surface. Accordingly, it was confirmed that the treatment with the extract mixture of Ziziphus jujuba Miller, Citrus unshiu Peel, and Acorus calamus had a hair health promotion effect.

The present disclosure has been described above with reference to preferred Examples and Experimental Examples thereof. It will be understood to those skilled in the art that the present disclosure may be implemented as modified forms without departing from an essential characteristic of the present disclosure. Therefore, the disclosed embodiments should be considered in an illustrative viewpoint rather than a restrictive viewpoint. The scope of the present disclosure is illustrated by the appended claims rather than by the foregoing description, and all differences within the scope of equivalents thereof should be construed as being included in the present disclosure.

What is claimed is:

1. A method for inhibiting inflammatory, oxidant, wrinkle, or improving hair health, the method comprising administering an extract mixture of Ziziphus jujuba Miller, Citrus unshiu Peel, and Acorus calamus to a subject in need thereof.

2. The method of claim 1, wherein the extract mixture is prepared by mixing 55 to 65 wt % of a Ziziphus jujuba Miller extract, 15 to 25 wt % of a Citrus unshiu Peel extract, and 15 to 25 wt % of an Acorus calamus extract.

3. The method of claim 2, wherein the Citrus unshiu Peel extract is prepared by adding distilled water 15 to 25 times as much as Citrus unshiu Peel powder, and then extracting and filtering the mixture at 10 to 30° C. for 20 to 28 hours.

4. The method of claim 2, wherein the Acorus calamus extract is prepared by adding distilled water 15 to 25 times as much as Acorus calamus powder, and then extracting and filtering the mixture at 7 to 100° C. for 20 to 28 hours.

5. The method of claim 1, wherein the subject requires inhibiting expression of MMP-1 and MMP-13.

6. The method of claim 1, wherein the subject requires inhibiting expression and production of IL-1β and IL-6.

7. The method of claim 1, wherein the subject requires increasing hair tensile strength.

8. A method for preventing or treating inflammatory or inhibiting oxidant, wrinkle, or improving hair health, the method comprising administering extract mixture of Ziziphus jujuba Miller, Citrus unshiu Peel, and Acorus calamus to a subject in need thereof.
